(19) United States
(12) Patent Application Publication
(10) Pub. No.: US 2026/0233018 A1
(43) Pub. Date: Aug. 13, 2026

(54) ELIMINATING AMYLOID PLAQUE AND TAU PROTEIN AND TANGLES BY WAY OF RESONANCE FREQUENCY OF MATTER

(71) Applicant: Jeffrey Allen Benz, Indianapolis, IN (US)

(72) Inventor: Jeffrey Allen Benz, Indianapolis, IN (US)

(21) Appl. No.: 19/537,161

(22) Filed: Feb. 11, 2026

Related U.S. Application Data
(60) Provisional application No. 63/757,151, filed on Feb. 11, 2025.

(51) Int. Cl. A61N 1/40; A61N 7/00

(52) U.S. Cl. CPC A61N 1/40; A61N 7/00; A61N 2007/0021

(57) ABSTRACT

The present invention provides a system and method to reverse dementia by eliminating amyloid plaque, mutated Tau Protein, and Tau Tangles. The system comprises a resonance/exciter device for generating resonance frequency matter to be utilized for eradicating the amyloid plaque, mutated Tau protein, and Tau tangles. Resonance excitation could be by means of RF (radio frequency) electromagnetic waves, sound waves, ultrasonics and mechanical means, such as MEMS. Through an HMI interface for control operations and parameters, the resonance generator/exciter will provide resonance frequency(s) to break up the amyloid plaque, mutated Tau protein, and Tau tangles into small particles and allow the excretory system within the brain to expel the small particles through the normal body processes.

### TECHNICAL FIELD

The present invention relates to a novel approach to eliminate amyloid plaque, mutated Tau Protein, and Tau Tangles. More particularly, the present invention relates to utilizing a physical property called resonance frequency of matter to eliminate amyloid plaque, mutated Tau Protein, and Tangles.

### BACKGROUND

In present times there has been a lot of interest in reducing or eliminating amyloid plaques, sometimes called Tau protein tangles. All founded research are using drugs for a means to slow down, reduce or eliminate amyloid plaque and Tau tangles. There has been some success in test drugs. Our approach is to eliminate the buildup and elimination of amyloid plaque, Tau tangles, and mutated Tau proteins by way of a physical property called resonance frequency of matter.

### SUMMARY

The present invention provides a system and a method of eliminating amyloid plaque, mutated Tau Protein, and Tangles by generating resonance frequency of matter which in this instance is resonance frequencies with the amyloid plaque, Tau tangles, and mutated Tau proteins so that we can break up these entities into small particles and allow the excretory system within the brain to expel the small particles through the normal body processes. This approach is through using non-invasive technology. The invention will reverse dementia and begin the ability to restore the rejuvenation and health of the neurons in the brain through normal body processes.

### DESCRIPTION OF THE DRAWING

FIG. 1. is a block diagram of the system for eliminating amyloid plaque, mutated Tau Protein, and Tangles;

FIG. 2. is a schematic view of the ablation process and what is targeted; and

FIG. 3. is a flow diagram showing the process and excretory of the ablated amyloid plaque, mutated Tau protein, and Tau tangles.

### DETAILED DESCRIPTIONS OF THE INVENTION

### Objective of the Invention

Every matter has a certain resonance frequency. Amyloid plaque, Tau tangles, and mutated Tua proteins are matters whose entities also possess the property of resonance. By determining the precise resonance frequency of these entities, we will be able to break up these entities into tiny particles. These tiny particles would be cleared away from the neuron axion (body) and more important the neuron endings (dendrites). By removing the amyloid plaque and Tau tangles from the dendrites we will allow the neuron to begin to heal and to function again. By removing the amyloid plaque and Tau tangles from the dendrites allows the synapses between the neurons to be restored and provide the means for the neurons to receive the required food and nutrients needed for restoration back to being a healthy neuron. This should return normal operations of the neuron and reverse dementia-like symptoms.

### Methods of Achieving Resonance Excitation

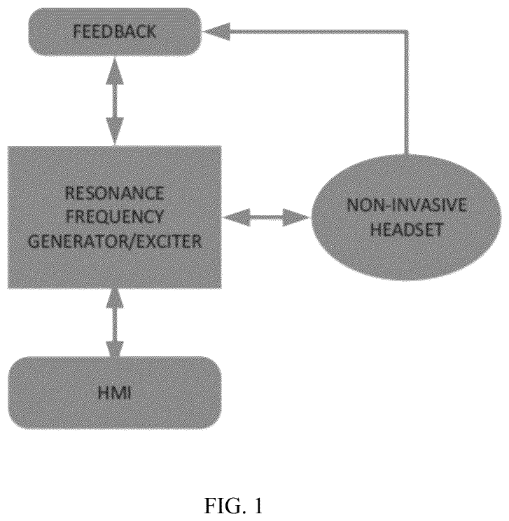

Referring to FIG. 1, illustrated is a block diagram of the system for eliminating amyloid plaque, mutated Tau Protein, and Tangles. There are countless ways of achieving resonance of a matter. Explorations into the way of providing resonance excitation could be by means of RF (radio frequency) electromagnetic waves, sound waves, ultrasonics, and mechanical means, such as micro-electromechanical system (MEMS). Because of the small mass of the makeup of the amyloidal plaque and Tau tangles we have been experimenting with both RF and ultrasonic means for resonance excitation. The sweeping frequencies of the resonance generator/exciter is in the high HF-UHF range of 250 MHz and 1 GHz, which covers the range of resonance frequencies of amyloid plaque, mutated Tau Protein, and Tangles. According to one of the embodiments, three generators and exciters are used simultaneously on one patient. According to other embodiments, more or less than three generators and exciters maybe used simultaneously on one patient.

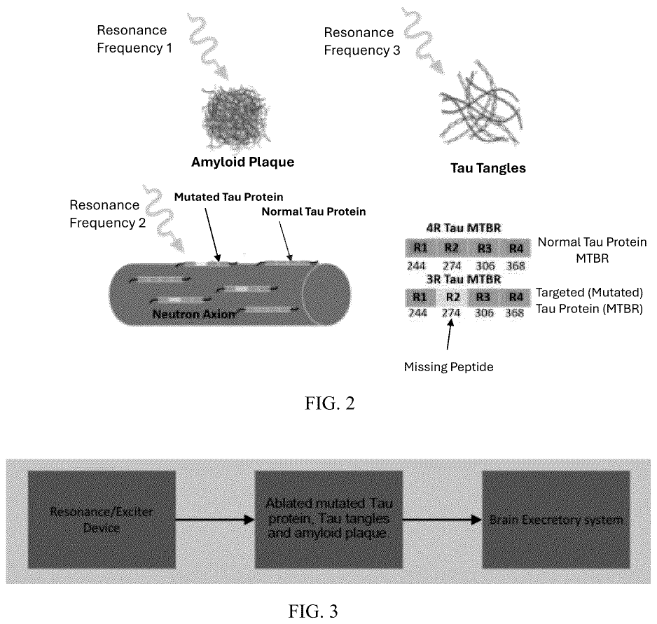

Referring to FIG. 2, illustrated is a schematic view of the ablation process and what is targeted. To target the proper amyloid plaque, Tau tangles, and mutated Tau proteins to break up or eliminate them, we know the normal Tau protein and the mutated Tau protein has different resonance frequencies. Thus, the excitation (resonance) energy of the desired frequency for the amyloid plague, mutated Tau protein, and Tangles will not affect the adjacent healthy cells. Since the amyloid plague, mutated Tau protein, and Tangles are very small the amount of resonance energy and the on-time period of the energy would be small. This technology would not require an energy source such as a CT scanner or MRI use.

The normal Tau protein has 3 main sections. Without going into many details, we are targeting the microtubule binding domain (MTBD). This section of the Tau protein causes the binding of the protein to the neuron axion. There are four peptides in this section in normal Tau protein. The mutated Tau protein has three peptides in this section and is the cause for the protein migration to the neuron's axion outer area and down to the dendrites producing what are called amyloid plaque and Tau tangles. This is the targeted Tau protein for what we term our process resonance ablation. Since this mutated Tau protein will have a different resonance frequency as the normal or good tau protein, we will be able to ablate or break up the mutated Tau protein without affecting the normal or good Tau protein.

### Invention Use

The present invention aims to reverse dementia using external sources and internal processes. The external source is derived from a medical device whereas it provides the necessary means to essentially eradicate the mutated Tau protein, Tau tangles, and amyloid plaque.

### Invention Operation

Through a human machine interface (HMI) for controlling operations and parameters the resonance generator/exciter will provide resonance frequency(s) to break up the amyloid plaque, mutated Tau protein, and Tau tangles.

### Components of Invention

The device, according to the present invention will accomplish this by applying resonance frequencies through a non-invasive means to the head through emitters such as antenna-resonators within a cap like device which is placed on the head. A feedback mechanism is used to monitor power, frequency characteristics, and other processes to ensure safety to both the healthcare provider and patient. The actual power levels in reference to energy are much less than cell phones, MRI, or CT scan energy devices. The device will operate in a carrier wave (CW), modulated or pulse mode for energy emissions. All control functions is through the HMI with the usage of a microcomputer, microprocessor, or any other type of software/firmware implemented components.

### Process of the Invention

The process of the present invention comprises a step of using resonance frequency(s) to ablate amyloid plaque, mutated Tau protein, and Tau tangles. This is facilitated by identifying presence of the amyloid plaque, mutated Tau protein, and Tau tangles within the head of a person. Since it has been determined that amyloid plaque, mutated Tau protein, and Tau tangles are one of the direct causes of dementia through multiple studies and research we need to be able to remove the amyloid plaque, mutated Tau protein, and Tau tangles. In this invention we target the amyloid plaque, mutated Tau protein, and Tau tangles using the physical properties of the amyloid plaque, mutated Tau protein, and Tau tangles to ablate.

It is also known that there are good Tau proteins and bad (mutated) Tau proteins. To explain the efficacy of the device we determine the resonance of the amyloid plaque, mutated Tau protein, and Tau tangles and introduce these frequencies into the head of the patient. Since the resonance frequency of the amyloid plaque, mutated Tau protein, and Tau tangles is uniquely different from that of the other proteins and other materials in the head, the other materials are not affected by the induced frequency(s) resonating with the amyloid plaque, mutated Tau protein, and Tau tangles. Through research the mutated Tau protein is missing the 2nd peptide within the Tau protein. Since this peptide is missing the resonance frequency is different from the normal Tau protein.

Referring to FIG. 3, illustrated is a flow diagram showing the process and excretory of the ablated amyloid plaque, mutated Tau protein, and Tau tangles. Step 1 of the process comprises installing a non-invasive means to the patient's head. The non-invasive means includes emitters such as antenna-resonators within a cap-like device which is placed on the head. Step 2 of the process comprises ablation of amyloid plaque, mutated Tau protein, and Tau tangles into small particles. Step 3 of the process comprises expelling small particles of amyloid plaque, mutated Tau protein, and Tau tangles from the brain excretory system.

What is claimed is:

1. A system for eliminating amyloid plaque, mutated Tau Protein, and Tau Tangles from head of a patient, the system comprising:
a non-invasive means, the non-invasive means including a resonance generator/exciter and a plurality of emitters, wherein the emitters are installed within a cap-like device placed on the head of the patient, and wherein the non-invasive means irradicates through the emitters sweeping frequencies resonant with a plaque in the head of the patient, the plaque selected from amyloid plaque, mutated Tau Protein, Tau Tangles, and a combination thereof;
a feedback mechanism used to monitor power, frequency characteristics, and other processes of the non-invasive means; and
a human machine interface (HMI) connected to the feedback mechanism.

2. The system of claim 1, wherein the human machine interface comprises a computer selected from a microcomputer, microprocessor, or any other type of software/firmware implemented components.

3. The system of claim 1, wherein the plurality of the emitters comprises antenna-resonators.

4. The system of claim 3, wherein the resonance generator/exciter is selected from a group consisting of RF (radio frequency) electromagnetic waves, sound waves, ultrasonics, and MEMS.

5. The system of claim 4, wherein the non-invasive means operates in a carrier wave (CW), the CW modulated for energy emissions.

6. The system of claim 4, wherein the non-invasive means operates in a carrier wave (CW) in a pulse mode for energy emissions.

7. A system for eliminating amyloid plaque, mutated Tau Protein, and Tau Tangles from head of a patient, the system comprising:
a non-invasive means, the non-invasive means including at least one resonance generator/exciter and a plurality of emitters, wherein the emitters are installed within a cap-like device placed on the head of the patient, wherein the plurality of the emitters comprises antenna-resonators, and wherein the non-invasive means irradicates through the emitters sweeping frequencies resonant with a plaque in the head of the patient.
a feedback mechanism used to monitor power, frequency characteristics, and other processes of the non-invasive means; and
a human machine interface (HMI) connected to the feedback mechanism.

8. The system of claim 7, wherein the plaque is selected from amyloid plaque, mutated Tau Protein, Tau Tangles, and a combination thereof.

9. The system of claim 8, wherein the human machine interface comprises a computer selected from a microcomputer, microprocessor, or any other type of software/firmware implemented components.

10. The system of claim 9, wherein the resonance generator/exciter is selected from a group consisting of RF (radio frequency) electromagnetic waves, sound waves, ultrasonics, and MEMS.

11. The system of claim 10, wherein the non-invasive means operates in a carrier wave (CW), the CW modulated for energy emissions.

12. The system of claim 10, wherein the non-invasive means operates in a carrier wave (CW) in a pulse mode for energy emissions.

13. A method of eliminating amyloid plaque, mutated Tau Protein, and Tangles from head of a patient, the method comprising:
identifying presence of amyloid plaque, mutated Tau protein, and Tau tangles in the head of a patient;
installing a non-invasive means to the patient's head, the non-invasive means including a resonance generator/exciter and a plurality of emitters, wherein the emitters are installed within a cap-like device placed on the head of the patient;
ablating plaque(s) in the head of the patient into small particles by using the non-invasive means through the emitters to irradiate sweeping frequencies resonant with the plaque, the plaque selected from amyloid plaque, mutated Tau Protein, Tau Tangles, and a combination thereof; and
expelling small particles of amyloid plaque, mutated Tau protein, and Tau tangles from the brain excretory system.

14. The method of claim 13, wherein power, frequency characteristics, and other processes of the non-invasive means are controlled by a feedback mechanism.

15. The method of claim 14, wherein the feedback mechanism is connected to a human machine interface (HMI).

16. The method of claim 15, wherein the human machine interface (HMI) comprises a computer selected from a microcomputer, microprocessor, or any other type of software/firmware implemented components.

17. The method of claim 16, wherein the resonance generator/exciter is selected from a group consisting of RF (radio frequency) electromagnetic waves, sound waves, ultrasonics, and MEMS.

18. The method of claim 17, wherein the resonance generator/exciter irradiates sweeping frequencies in the high HF-UHF range of 250 MHz and 1 GHz.

19. The method of claim 18, wherein the non-invasive means operates in a carrier wave (CW), the CW modulated for energy emissions.

20. The method of claim 18, wherein the non-invasive means operates in a carrier wave (CW) in a pulse mode for energy emissions.
